(19) United States
(12) Patent Application Publication
(10) Pub. No.: US 2020/0345543 A1
(43) Pub. Date: Nov. 5, 2020

(54) REAL-TIME TREATMENT ADJUSTMENT BASED ON DOSIMETRIC DATA

(71) Applicant: Xinova, LLC, Seattle, WA (US)

(72) Inventor: Ezekiel KRUGLICK, Poway, CA (US)

(73) Assignee: Xinova, LLC, Seattle, WA (US)

(21) Appl. No.: 16/962,862

(22) PCT Filed: Feb. 2, 2018
(86) PCT No.: PCT/US18/16547

§ 371 Date: Jul. 17, 2020

(51) Int. Cl. A61F 9/008

(52) U.S. Cl. CPC A61F 9/008; A61F 2009/00897; A61F 2009/0088

(57) ABSTRACT

Technologies are generally described for customization of treatment based on dosimetric data collected during the treatment. In some examples, a laser treatment procedure may involve the application of multiple laser pulses to a treatment site. During the laser treatment procedure, an effect resulting from the application of one or more of the laser pulses may result in dosimetric data, such as acoustic and/or optical data. The dosimetric data may then be used to determine the efficacy of the laser treatment procedure and/or to adjust, in real-time, a remainder of the laser treatment procedure.

### BACKGROUND

Unless otherwise indicated herein, the materials described in this section are not prior art to the claims in this application and are not admitted to be prior art by inclusion in this section.

The efficacy of medical procedures and treatments necessarily varies based upon the individual patient. For example, a particular procedure or treatment may work relatively well for certain individuals, but not for other individuals. The degree to which a procedure or treatment is suitable for an individual may be based on characteristics of the procedure or treatment and characteristics of the individual.

### SUMMARY

The present disclosure generally describes techniques for real-time treatment adjustment based on dosimetric data.

According to some examples, a method is provided to personalize a laser treatment procedure on a patient. The method may include, in response to application of a first laser pulse to a treatment site of the patient as part of the laser treatment procedure, receiving a signal based on an observation of an effect of the first laser pulse. The method may further include determining a classification for the patient based on the received signal, adjusting a remainder of the laser treatment procedure based on the classification of the patient, and continuing the adjusted remainder of the laser treatment procedure.

According to other examples, an apparatus to personalize a laser treatment procedure on a patient is provided. The apparatus may include a detection device and a processor coupled to the detection device. The detection device may be configured to detect an effect of a first laser pulse in response to a treatment site of the patient as part of the laser treatment procedure, and generate a signal based on the detected effect. The processor may be configured to receive the signal from the detection device, determine a classification for the patient based on the received signal, determine an adjustment for a remainder of the laser treatment procedure based on the classification of the patient, and provide the adjustment for the remainder of the laser treatment procedure to a laser treatment system and/or a healthcare personnel.

According to further examples, a system is provided to personalize a laser treatment procedure on a patient. The system may include a laser device, a detection device, and a processor coupled to the laser device and the detection device. The laser device may be configured to provide multiple laser pulses to a treatment site of the patient as part of the laser treatment procedure. The detection device may be configured to detect an effect of a first laser pulse at the treatment site in response to application of the first laser pulse by the laser device, and generate a signal based on the detected effect. The processor may be configured to receive the signal from the detection device, determine a classification for the patient based on the received signal, determine an adjustment for a remainder of the laser treatment procedure based on the classification of the patient, and provide the adjustment for the remainder of the laser treatment procedure to the laser device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example treatment system where real-time treatment customization based on dosimetric data may be implemented;

FIG. 2 illustrates an example laser ophthalmological treatment system where real-time treatment customization based on dosimetric data may be implemented;

FIG. 3 illustrates how reference dosimetric data may be used to generate patient categories;

FIG. 4 is a flowchart illustrating an example laser ophthalmological process involving patient classification and treatment customization based on dosimetric data;

FIG. 5 illustrates a computing device, which may be used to provide real-time treatment customization based on dosimetric data;

FIG. 6 is a flow diagram illustrating an example method to perform real-time treatment customization based on dosimetric data that may be performed by a computing device such as the computing device in FIG. 5; and

FIG. 7 illustrates a block diagram of an example computer program product, all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to real-time treatment customization based on dosimetric data.

Briefly stated, technologies are generally described for customization of treatment based on dosimetric data collected during the treatment. In some examples, a laser treatment procedure may involve the application of multiple laser pulses to a treatment site. During the laser treatment procedure, an effect resulting from the application of one or more of the laser pulses may result in dosimetric data, such as acoustic and/or optical data. The dosimetric data may then be used to determine the efficacy of the laser treatment procedure and/or to adjust, in real-time, a remainder of the laser treatment procedure.

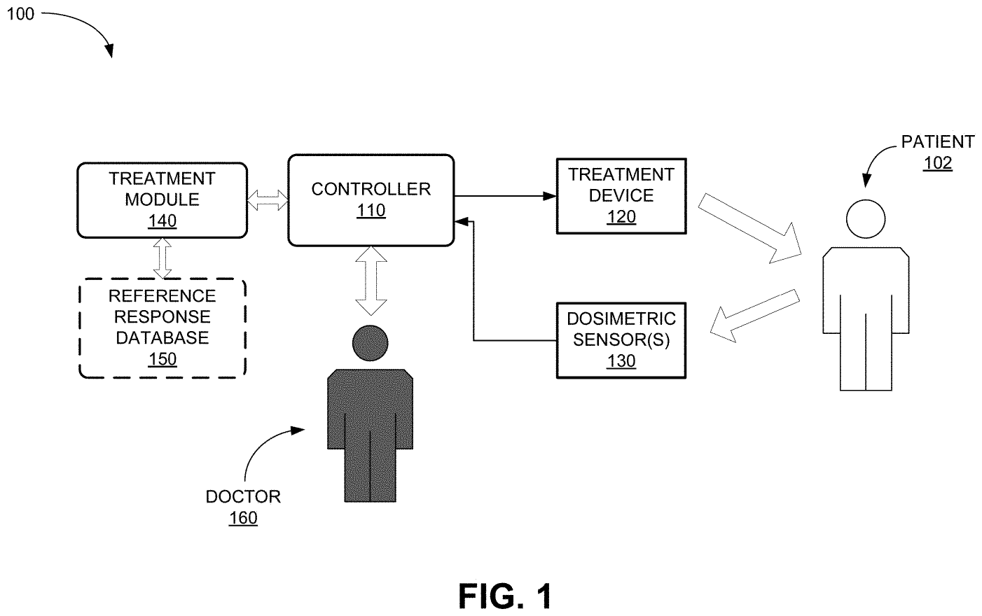

FIG. 1 illustrates an example treatment system 100 where real-time treatment customization based on dosimetric data may be implemented, arranged in accordance with at least some embodiments described herein.

The treatment system 100 may include a controller 110, a treatment device 120, one or more dosimetric sensors 130, a treatment module 140, and an optional reference response database 150, and may be configured to perform treatment on a patient 102. The treatment may be any treatment suitable for the patient 102, such as a medical treatment, a cosmetic treatment, or any other suitable treatment. The treatment device 120 may be configured to perform treatment on the patient 102 in response to control signals from the controller 110. The dosimetric sensor(s) 130 may be configured to sense dosimetric data from the patient 102 resulting from observed effects of the performed treatment and transmit the sensed data to the controller 110. The controller 110 may then provide the sensed data to the treatment module 140. The treatment module 140, in turn, may be configured to use the sensed data to determine the efficacy of the performed treatment and/or adjustments for the treatment to increase efficacy, avoid damage, or for any suitable rationale.

In an example scenario, the laser treatment procedure may be surgical treatment of melanosomes, an abnormal growth on the retina of an eye. Thus, the target treatment area may be a portion of the retina. The applied laser beams may generate heat at the treatment site, which in turn may result in formation of bubbles (through the expansion of fluids within the diseases cells transforming into gases) on the retina. The physical response (formation of the bubbles) may be detected acoustically through detection of pressure waves in vitreous fluid (through a probe physically touching a surface of the eye, for example) or optically (through Doppler interferometry or reflectometry based on relatively large refractive index difference between bubbles and the surrounding fluid).

In some example embodiments, the treatment module 140 may use the sensed data to determine some characteristic or classification of the patient 102, and may adjust the treatment based on the determined characteristic/classification. In some embodiments, the treatment module 140 may communicate with a reference response database 150 to determine the characteristic and/or classification of the patient 102. For example, the reference response database 150 may store information about how patient 102 and/or other patients have responded to treatments with different parameters, information about how certain patient parameters or characteristics affect treatment, or any other suitable data relevant to the treatment. The treatment module 140, in response to determining adjustments to the treatment, may provide the adjustments to the controller 110, which may then actuate the treatment device 120 accordingly. In some embodiments, a doctor 160 or other supervisory entity may interact with the controller 110 as a check or fail-safe in order to ensure that the adjustments provided by the treatment module 140 are in fact suitable.

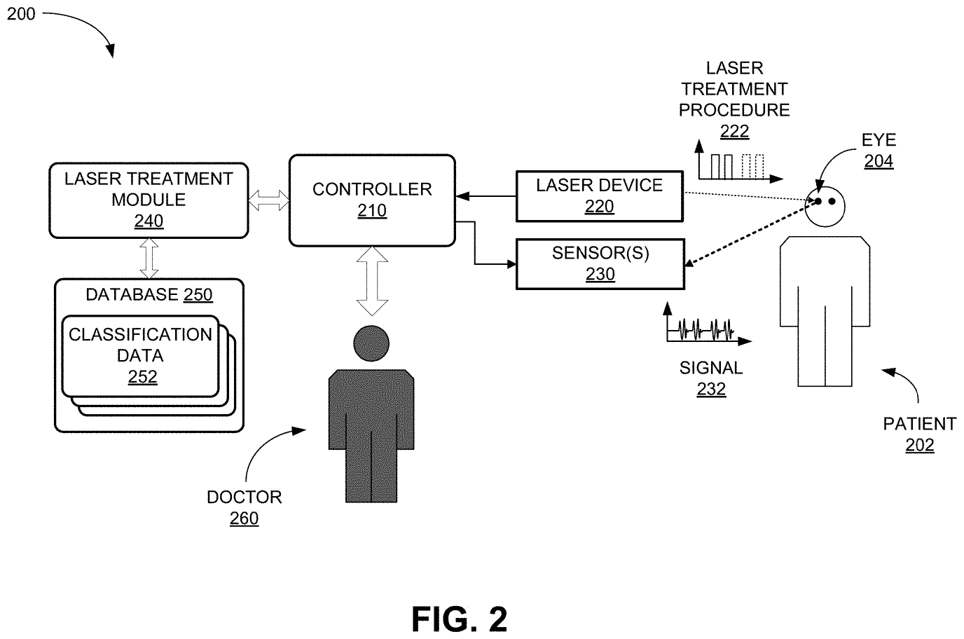

FIG. 2 illustrates an example laser ophthalmological treatment system 200 where real-time treatment customization based on dosimetric data may be implemented, arranged in accordance with at least some embodiments described herein.

The laser ophthalmological treatment system 200 is similar to the treatment system 100 in general operation. The laser ophthalmological treatment system 200 may include a controller 210, a laser device 220, one or more sensors 230, a laser treatment module 240, and a database 250 to store classification data 252.

The laser device 220 may be configured to perform procedure 222 on a patient 202 in response to control signals from the controller 210. The laser device 220 may include a laser source or generator and a laser controller, and may be configured to generate and direct laser energy at or into an eye 204 of the patient 202. The laser device 220 may be configured to perform the laser treatment 222 using a continuous laser beam, or via a series of laser pulses. In some embodiments, the laser treatment procedure 222 may involve the application of laser energy to some portion of the eye 204 in order to generate heat and cauterize tissue.

The sensor(s) 230 may be configured to sense signal(s) 232 from the patient 202 and/or the eye 204 resulting from at least part of the laser treatment procedure 222. The signals 232 may be associated with observed effects of the laser treatment procedure 222, and may include acoustic data, optical data, reflectometry data, electromagnetic data, interferometric data, any other suitable data related to the laser treatment procedure 222, or a combination of the foregoing such as acousto-optic data. Acoustic data may be related to the effect of acoustic energy originating from the generation of heat in tissues of the eye 204. Optical data or reflectometry data may be related to the effect of optically-visible changes to tissues of the eye 204 due to the laser treatment procedure 222. The sensor(s) 230 may then be configured to send the signals 232 to the controller 210.

The controller 210 may be configured to provide control signals to the laser device 220 to cause the performance of laser treatment procedure 222. For example, the controller 210 may provide control signals that indicate the amplitude and/or frequency of the laser energy to be generated. The control signals may also or instead indicate whether the laser device 220 is to generate a continuous laser beam or laser pulses, and if the latter the number of pulses, the strengths or shapes of the individual pulses, pulse timing parameters such as pulse duration, pulse generation time, pulse spacing, and any other suitable laser parameter. Further examples of laser parameters that may be adjusted, may include pulse shape (modulation), beam profile, radiance, intensity, energy, and comparable properties associated with the applied laser beam and/or laser pulses. In some embodiments, the controller 210 may provide a program or profile to the laser device 220, and a controller within the laser device 220 may be responsible for determining appropriate laser generation parameters based on the provided program or profile.

In some embodiments, the controller 210 may determine the control signals based on treatment information received from the laser treatment module 240. As described above, the sensor(s) 230 may be configured to send the signals 232 to the controller 210. The controller 210 may then send the signals 232 to the laser treatment module 240, which in turn may use the signals 232 to determine whether adjustments are to be made to the laser treatment procedure 222, and if so to determine the appropriate adjustments. In some embodiments, the laser treatment module 240 compares the signals 232 to data (for example, the classification data 252) stored in the database 250. The database 250 may be a local database (for example, co-located with the laser treatment module 240) or a remote database (for example, located at a separate facility), and may store classification data 252 as well as other patient- and treatment-relevant data. The classification data 252 may include or be based on reference dosimetric data associated with past patients, laser treatment procedures, and their outcomes. By comparing the signals 232 with the classification data 252, the laser treatment module 240 may be able to determine the likely outcome of the laser treatment procedure 222, as well as adjustments that can be made to the laser treatment procedure 222 to increase the probability of a successful outcome. In some embodiments, the laser treatment module 240 may use the signals 232 and the classification data 252 to classify the patient 202 and/or the eye 204 into one of multiple categories, and then customize the laser treatment procedure 222 based on the category. In some embodiments, the laser treatment module 240 may classify the patient 202 and/or the eye 204 into multiple categories simultaneously based on the signals 232, especially if the signals 232 include distinct types of signals or signals from different sensors, and may then customize or adjust the laser treatment procedure 222 based on the categories.

In response to a determination of appropriate adjustments (if any) to the laser treatment procedure 222, the laser treatment module 240 may then send the adjustments back to the controller 210, which in turn may send control signals to the laser device 220 to adjust the laser treatment procedure 222 in real-time (in other words, during the laser treatment procedure 222). Such adjustments may include adjustments to the frequency and/or amplitude of the laser generated by the laser device 220, and if the laser treatment procedure 222 involves the application of multiple laser pulses, the adjustments may include the remaining number of pulses to be generated, the strength or shape of the remaining pulses, and/or the timing as to when the remaining pulses or when individual pulses are to be generated. The adjustments may also include beam profile, radiance, intensity, energy, and comparable properties associated with the applied laser beam and/or laser pulses. A doctor 260 or some other supervisory entity may be present to act as a check or fail-safe on the laser ophthalmological treatment system 200, to approve the adjustments provided by the laser treatment module 240.

As described above, classification data used to classify patients may include or be based on reference dosimetric data associated with past patients, laser treatment procedures, and their outcomes. In some embodiments, the reference dosimetric data for each patient may take the form of time-magnitude data traces, for example of acoustic and/or optical signals. To generate classification data, a distance (or the inverse, similarity) metric may be computed between the reference dosimetric data for each pair of patients. In some embodiments, the distance metric may be computed using a dynamic time warping technique and/or a wavelet technique, although any other technique to compute distance or similarity between two data sets may be used. The computed distance metrics may then be used to classify patients into different groups or categories, where pairs of patients with relatively low distance metrics (or relatively high similarity metrics) are more likely to be in the same group than pairs of patients with relatively high distance metrics. In determining the classification, a series of categories and a difference metric to determine the difference between the collected data and various references for each category may be used, and a category selected based on minimum difference from the collected data (e.g., distance metric). In some examples, scoring metrics may be generated and grouping performed using “greater than” or “less than” rules, for example, by a successive series of comparisons in a decision tree. In other examples, an ensemble classifier may be used, for example, generating variously weighted multiple decision trees and/or difference metrics.

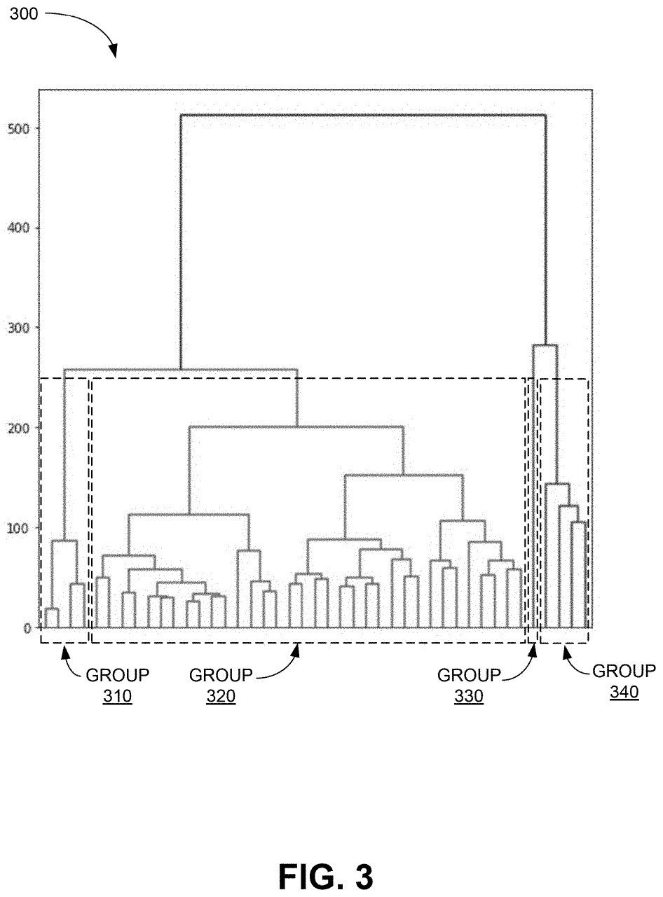

FIG. 3 illustrates how reference dosimetric data may be used to generate patient categories, arranged in accordance with at least some embodiments described herein.

Chart 300 depicts a dendrogram showing the similarity of sets of example reference dosimetric data corresponding to different patients, with the horizontal axis representing the different patients and the vertical axis representing an arbitrary distance metric. In chart 300, the patients are ordered such that the most similar patients are adjacent to each other. The height along the vertical axis at which a path connects two patients may represent the distance metric between the two patients. The patients may then be grouped into a number of categories or bins based on a desired threshold distance metric. For example, as depicted in chart 300, generating patient groups based on a threshold distance metric of 250 may result in four different groups 310, 320, 330, and 340. Generating patient groups based on a lower threshold distance metric may tend to result in more groups, whereas generating patient groups based on a higher threshold distance metric may tend to result in fewer groups. Groups may represent eye elasticity associated with age, different medical conditions, scarring from previous procedures, different genetic or structural types, etc. Groups discovered numerically may then be combined with medical review to determine the actual phenomena or patient types being measured, which may be used to refine or further define the groups.

When a treatment module such as the treatment module 140 or the laser treatment module 240 receives sensed dosimetric data associated with a patient undergoing treatment, the treatment module may compute distance metrics between the sensed dosimetric data and one or more sets of reference dosimetric data and use the computed distance metrics to classify the patient into a group. In some embodiments, each group may be associated with a set of similarity parameters, and the treatment module may extract corresponding parameters from the sensed dosimetric data and compare the extracted parameters to the group similarity parameters to identify a group within which the patient should be classified, for example based on the extent to which the extracted parameters differ from the group similarity parameters. In some embodiments, patient classification may occur based on a successive series of comparisons of the sensed dosimetric data to reference dosimetric data in a decision tree. For example, if a computed difference or distance between the sensed dosimetric data and reference dosimetric data associated with a particular category or group is below one or more thresholds, the patient may be classified within that category or group, whereas if the computed difference or distance is greater than the threshold(s), the patient may not be classified within that category or group, and instead may be compared to one or more other categories or groups. Other estimators or classification techniques, such as ensemble classification, support vector classification, k-nearest-neighbors classification, stochastic gradient descent classification, kernel approximation classification, any other suitable classification techniques, or a combination of one or more of the previous, may be used for patient classification. In other embodiments, any other technique for determining the similarity of sensed dosimetric data to reference dosimetric data or patient groups may be used.

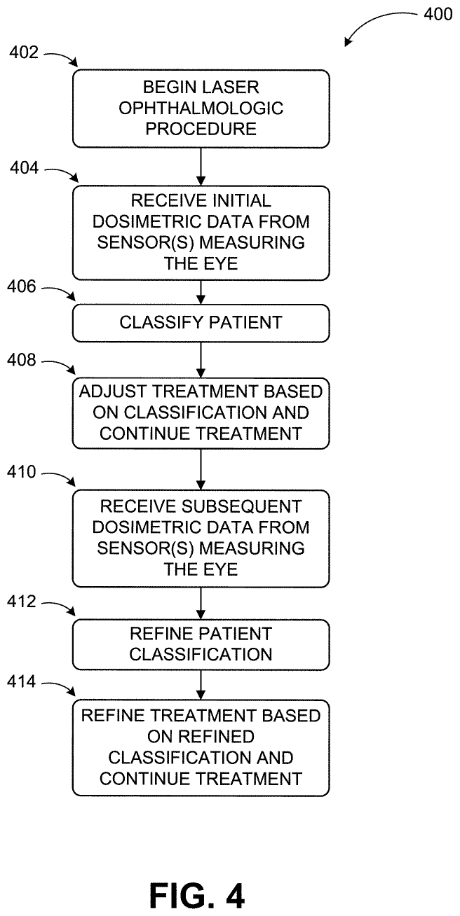

FIG. 4 is a flowchart illustrating an example laser ophthalmological process 400 involving patient classification and treatment customization based on dosimetric data, arranged in accordance with at least some embodiments described herein.

The process 400 may begin at block 402 (“Begin laser ophthalmologic procedure”), where a laser ophthalmological treatment system initiates a procedure involving the application of laser energy, in a continuous beam or in pulses. The laser energy may be applied to a treatment site of a patient, such as the eye 204 of the patient 202.

At block 404 (“Receive initial dosimetric data from sensor(s) measuring the eye”), which may follow block 402, the laser ophthalmological treatment system may receive dosimetric data from one or more sensors (for example, the sensors 230) from the patient eye resulting from the initial application of laser energy. The dosimetric data may be acoustic data, optical data, reflectometry data, electromagnetic data, interferometric data, a combination of multiple dosimetric data types, or any other suitable dosimetric data.

At block 406 (“Classify patient”), which may follow block 404, the laser ophthalmological treatment system may use the initial dosimetric data received at block 404 to classify the patient into one or more patient groups or categories, as described above. In some embodiments, data other than dosimetric data may be used to classify the patient or to inform additional treatment. For example, characteristics of the patient eye, such as a size of the eye, an elasticity of the eye, a pressure of the eye, a fluid content of the eye, a location of a photoceptor cell at the treatment site, a position of the photoceptor cell at the treatment site, a type of the photoceptor cells at the treatment site, an amount of melanin at the treatment site, or a content of stem cells near the treatment site, and/or any other parameter or characteristic associated with the eye or patient, may be used to classify the patient or determine treatment adjustments. In some embodiments, the laser ophthalmological treatment system may perform the classification with some associated confidence metric that indicates the likelihood that the classification is appropriate.

At block 408 (“Adjust treatment based on classification and continue treatment”), which may follow block 406, the laser ophthalmological treatment system may use the classification at block 406 to adjust the treatment process, and continue the treatment process. For example, the laser ophthalmological treatment system may adjust a frequency or amplitude of the laser beam, a number of laser pulses remaining in the treatment process, a duration or width of the individual laser pulses remaining in the treatment process, a strength or shape of the laser pulses remaining in the treatment process, a time separation between adjacent laser pulses remaining in the treatment process, or any other parameter associated with the treatment process such as pulse shape (modulation), beam profile, radiance, intensity, energy, and comparable properties associated with the applied laser beam and/or laser pulses. In some embodiments, the laser ophthalmological treatment system may use the confidence metric determined at block 406 to inform the degree to which the treatment process is adjusted.

At block 410 (“Receive subsequent dosimetric data from sensor(s) measuring the eye”), which may follow block 408, the laser ophthalmological treatment system may receive additional dosimetric data from the sensors from which initial dosimetric data was received at block 404. The additional dosimetric data may be sensed in response to additional application of laser energy in the treatment process.

At block 412 (“Refine patient classification”), which may follow block 410, the laser ophthalmological treatment system may use the additional dosimetric data received at block 410, alone or in combination with the initial dosimetric data received at block 404 or other, prior dosimetric data, to refine the classification of the patient if necessary. For example, the laser ophthalmological treatment system may only use the additional dosimetric data, may use an average of the initial, additional, and/or other dosimetric data, may use a difference between the initial, additional, and/or other dosimetric data, may use a rolling window including some of the initial, additional, and/or other dosimetric data, or may use some other combination of the initial, additional, and other dosimetric data, to refine the patient classification.

At block 414 (“Refine treatment based on refined classification and continue treatment”), which may follow block 412, the laser ophthalmological treatment system may use the patient classification refined in block 412 to further adjust the treatment process, and may continue the treatment process. In some embodiments, the laser ophthalmological treatment system may halt the treatment process based on the refined patient classification. In other embodiments, the laser ophthalmological treatment system may repeat the cycle of receiving additional dosimetric data (for example, block 410), refining patient classification (for example, block 412), and refining the treatment based on the refined patient classification (for example, block 414), until the treatment is complete or otherwise halted.

The examples provided in FIGS. 1 through 4 are illustrated with specific systems and processes. Embodiments are not limited to environments according to these examples. Real-time customization of treatment processes may be implemented in environments employing fewer or additional systems and scenarios. For example, real-time customization based on dosimetric data may be implemented for other medical procedures and processes in addition to laser ophthalmology treatment. Furthermore, the example systems and processes shown in FIGS. 1 through 4 may be implemented in a similar manner with other user interface or action flow sequences using the principles described herein.

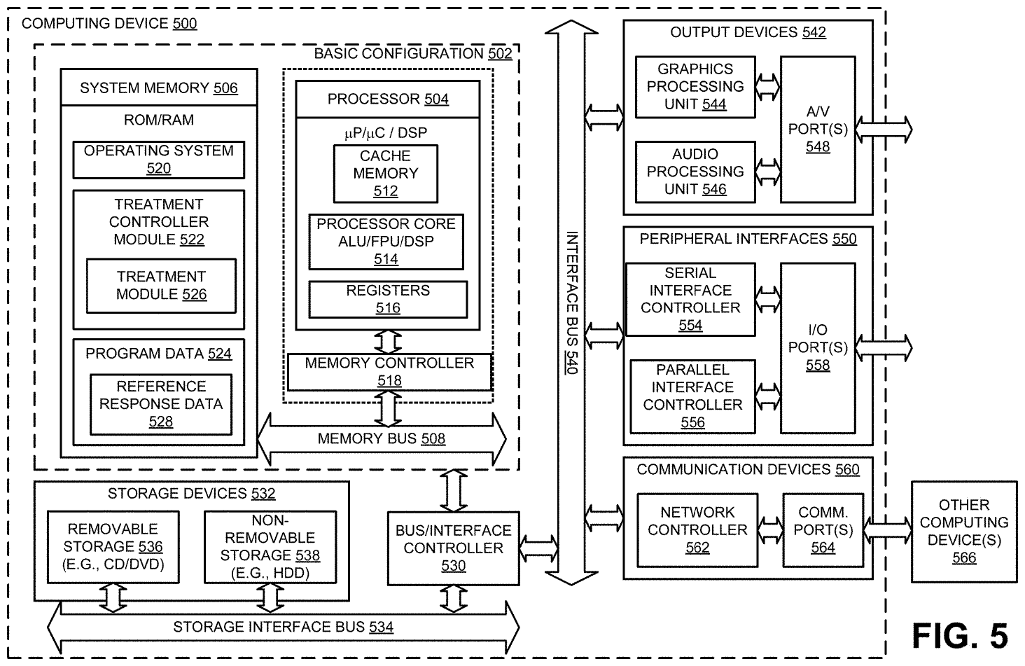

FIG. 5 illustrates a computing device, which may be used to provide real-time treatment customization based on dosimetric data, arranged in accordance with at least some embodiments described herein.

For example, the computing device 500 may be used to customize laser treatment procedure in real-time based on dosimetric data. In an example basic configuration 502, the computing device 500 may include one or more processors 504 and a system memory 506. A memory bus 508 may be used to communicate between the processor 504 and the system memory 506. The basic configuration 502 is illustrated in FIG. 5 by those components within the inner dashed line.

Depending on the desired configuration, the processor 504 may be of any type, including but not limited to a microprocessor (μP), a microcontroller (μC), a digital signal processor (DSP), or any combination thereof. The processor 504 may include one or more levels of caching, such as a cache memory 512, a processor core 514, and registers 516. The example processor core 514 may include an arithmetic logic unit (ALU), a floating point unit (FPU), a digital signal processing core (DSP core), or any combination thereof. An example memory controller 518 may also be used with the processor 504, or in some implementations, the memory controller 518 may be an internal part of the processor 504.

Depending on the desired configuration, the system memory 506 may be of any type including but not limited to volatile memory (such as RAM), non-volatile memory (such as ROM, flash memory, etc.) or any combination thereof. The system memory 506 may include an operating system 520, a treatment controller module 522, and program data 524. The treatment controller module 522 may include a treatment module 526 configured to perform real-time customization of treatment based on dosimetric data as described herein. The program data 524 may include, among other data, reference response data 528 or the like, as described herein.

The computing device 500 may have additional features or functionality, and additional interfaces to facilitate communications between the basic configuration 502 and any desired devices and interfaces. For example, a bus/interface controller 530 may be used to facilitate communications between the basic configuration 502 and one or more data storage devices 532 via a storage interface bus 534. The data storage devices 532 may be one or more removable storage devices 536, one or more non-removable storage devices 538, or a combination thereof. Examples of the removable storage and the non-removable storage devices include magnetic disk devices such as flexible disk drives and hard-disk drives (HDDs), optical disk drives such as compact disc (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 506, the removable storage devices 536 and the non-removable storage devices 538 are examples of computer storage media. Computer storage media includes, but is not limited to, RAM, ROM, EEPROM, flash memory or other memory technology, CD-ROM, digital versatile disks (DVDs), solid state drives (SSDs), or other optical storage, magnetic cassettes, magnetic tape, magnetic disk storage or other magnetic storage devices, or any other medium which may be used to store the desired information and which may be accessed by the computing device 500. Any such computer storage media may be part of the computing device 500.

The computing device 500 may also include an interface bus 540 for facilitating communication from various interface devices (e.g., one or more output devices 542, one or more peripheral interfaces 550, and one or more communication devices 560) to the basic configuration 502 via the bus/interface controller 530. Some of the example output devices 542 include a graphics processing unit 544 and an audio processing unit 546, which may be configured to communicate to various external devices such as a display or speakers via one or more A/V ports 548. One or more example peripheral interfaces 550 may include a serial interface controller 554 or a parallel interface controller 556, which may be configured to communicate with external devices such as input devices (e.g., keyboard, mouse, pen, voice input device, touch input device, etc.) or other peripheral devices (e.g., printer, scanner, etc.) via one or more I/O ports 558. An example communication device 560 includes a network controller 562, which may be arranged to facilitate communications with one or more other computing devices 566 over a network communication link via one or more communication ports 564. The one or more other computing devices 566 may include servers at a datacenter, customer equipment, and comparable devices.

The network communication link may be one example of a communication media. Communication media may be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 500 may be implemented as a part of a general purpose or specialized server, mainframe, or similar computer that includes any of the above functions. The computing device 500 may also be implemented as a personal computer including both laptop computer and non-laptop computer configurations.

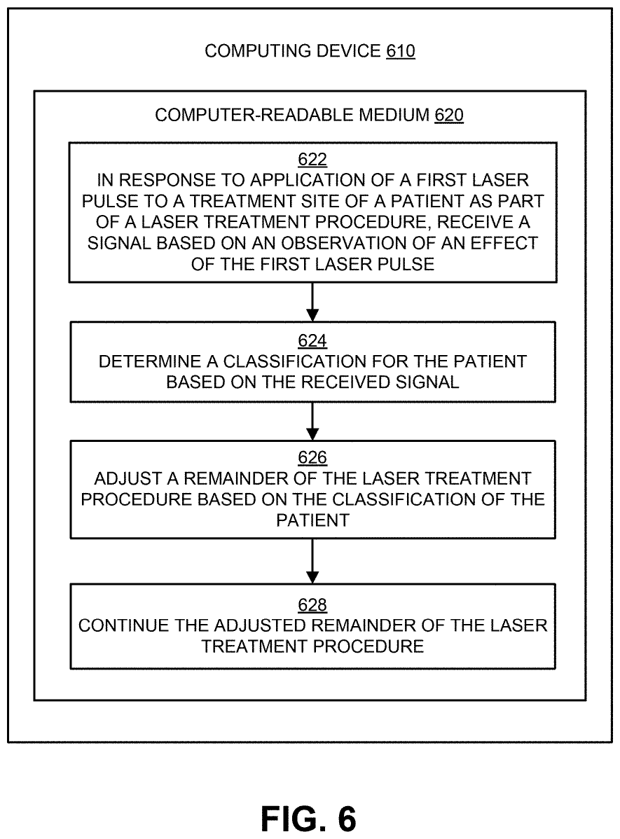

FIG. 6 is a flow diagram illustrating an example method to perform real-time treatment customization based on dosimetric data that may be performed by a computing device such as the computing device in FIG. 5, arranged in accordance with at least some embodiments described herein.

Example methods may include one or more operations, functions or actions as illustrated by one or more of blocks 622, 624, 626, and/or 628, and may in some embodiments be performed by a computing device such as the computing device 610 in FIG. 6. Such operations, functions, or actions, in FIG. 6 and in the other figures, in some embodiments, may be combined, eliminated, modified, and/or supplemented with other operations, functions, or actions, and need not necessarily be performed in the exact sequence as shown. The operations described in the blocks 622-628 may also be implemented through execution of computer-executable instructions stored in a computer-readable medium such as a computer-readable medium 620 of a computing device 610.

An example process to perform real-time customization of laser treatment may begin with block 622, “IN RESPONSE TO APPLICATION OF A FIRST LASER PULSE TO A TREATMENT SITE OF A PATIENT AS PART OF A LASER TREATMENT PROCEDURE, RECEIVE A SIGNAL BASED ON AN OBSERVATION OF AN EFFECT OF THE FIRST LASER PULSE”, where a laser treatment system may receive a signal associated with an observation of an effect of a first laser pulse applied to a patient treatment site as part of a laser treatment procedure, as described above. The signal may include an acoustic signal, an optical signal, a reflectometry signal, or a combination of different signal types. The “first” laser pulse as used herein may not necessarily be the first laser pulse of the treatment, but may be the first laser pulse of the measurement. For example, the measurement may begin after the second, third, or later laser pulses in the treatment.

Block 622 may be followed by block 624, “DETERMINE A CLASSIFICATION FOR THE PATIENT BASED ON THE RECEIVED SIGNAL”, where, during the treatment process, the laser treatment system may use the signal to classify the patient into a patient group, as described above. In some embodiments, the laser treatment system may perform the classification by comparing the signal to reference dosimetric data. The laser treatment system may also use other data about the patient or the treatment site to perform the classification.

Block 624 may be followed by block 626, “ADJUST A REMAINDER OF THE LASER TREATMENT PROCEDURE BASED ON THE CLASSIFICATION OF THE PATIENT”, where the laser treatment system may use the classification to adjust the rest of the laser treatment procedure. For example, the laser treatment system may adjust a duration, a time spacing, shape (modulation), beam profile, radiance, intensity, energy, and comparable properties associated with the applied laser beam and/or laser pulses remaining in the procedure.

Block 626 may be followed by block 628, “CONTINUE THE ADJUSTED REMAINDER OF THE LASER TREATMENT PROCEDURE”, where the laser treatment system may proceed to continue the laser treatment procedure with the adjustments of block 626. In some embodiments, the adjustments may involve halting the laser treatment procedure, in which case the laser treatment system may halt the procedure.

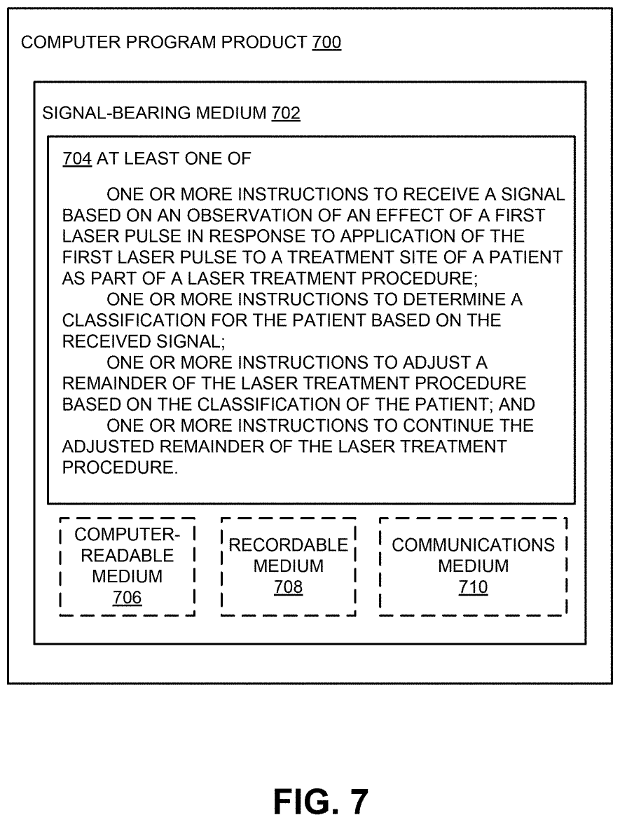

FIG. 7 illustrates a block diagram of an example computer program product, arranged in accordance with at least some embodiments described herein.

In some examples, as shown in FIG. 7, a computer program product 700 may include a signal-bearing medium 702 that may also include one or more machine readable instructions 704 that, when executed by, for example, a processor may provide the functionality described herein. Thus, for example, referring to the processor 504 in FIG. 5, the treatment controller module 522 may undertake one or more of the tasks shown in FIG. 7 in response to the instructions 704 conveyed to the processor 504 by the signal-bearing medium 702 to perform actions associated with treatment customization as described herein. Some of those instructions may include, for example, instructions to receive a signal based on an observation of an effect of a first laser pulse in response to application of the first laser pulse to a treatment site of a patient as part of a laser treatment procedure, determine a classification for the patient based on the received signal, adjust a remainder of the laser treatment procedure based on the classification of the patient, and/or continue the adjusted remainder of the laser treatment procedure, according to some embodiments described herein.

In some implementations, the signal-bearing medium 702 depicted in FIG. 7 may encompass computer-readable medium 706, such as, but not limited to, a hard disk drive (HDD), a solid state drive (SSD), a compact disc (CD), a digital versatile disk (DVD), a digital tape, memory, etc. In some implementations, the signal-bearing medium 702 may encompass recordable medium 708, such as, but not limited to, memory, read/write (R/W) CDs, R/W DVDs, etc. In some implementations, the signal-bearing medium 702 may encompass communications medium 710, such as, but not limited to, a digital and/or an analog communication medium (e.g., a fiber optic cable, a waveguide, a wired communication link, a wireless communication link, etc.). Thus, for example, the computer program product 700 may be conveyed to one or more modules of the processor 504 by an RF signal-bearing medium, where the signal-bearing medium 702 is conveyed by the communications medium 710 (e.g., a wireless communications medium conforming with the IEEE 802.11 standard).

According to some examples, a method is provided to personalize a laser treatment procedure on a patient. The method may include, in response to application of a first laser pulse to a treatment site of the patient as part of the laser treatment procedure, receiving a signal based on an observation of an effect of the first laser pulse. The method may further include determining a classification for the patient based on the received signal, adjusting a remainder of the laser treatment procedure based on the classification of the patient, and continuing the adjusted remainder of the laser treatment procedure.

According to some embodiments, determining the classification for the patient may include determining a category for the patient based on the effect of the first laser pulse among multiple categories, where the multiple categories are based on collected data from multiple patients. Adjusting the remainder of the laser treatment procedure may include adjusting a number of remaining laser pulses for a completion of the laser treatment procedure, adjusting a strength of remaining laser pulses for a completion of the laser treatment procedure, and/or adjusting a timing of remaining laser pulses for a completion of the laser treatment procedure. Receiving the signal based on the observation of the effect of the first laser pulse may include receiving the signal based on an acoustic detection or an optical detection of the effect of the first laser pulse, and/or receiving multiple signal types.

According to other embodiments, determining the classification for the patient may include computing a similarity metric between the signal and a reference signal and determining the classification for the patient based on the similarity metric. Computing the similarity metric may include computing the similarity metric through a dynamic time warping technique and/or through a wavelet technique. The treatment site is an eye and determining the classification for the patient further includes determining the category for the patient based on the effect of the first laser pulse and a characteristic of the eye. The characteristic may include a size of the eye, an elasticity of the eye, a pressure of the eye, a fluid content of the eye, a location of a photoceptor cell at the treatment site, a position of the photoceptor cell at the treatment site, a type of the photoceptor cells at the treatment site, an amount of melanin at the treatment site, or a content of stem cells near the treatment site.

According to further embodiments, the method may further include determining a confidence metric for the classification of the patient based on the signal. The method may further include receiving an other signal based on the observation of an effect of a second laser pulse in response to application of the second laser pulse to the treatment site of the patient as part of the laser treatment procedure, and determining the classification for the patient based on the signal and the other signal. Determining the classification for the patient based on the signal and the other signal may include determining the classification for the patient based on an average of the signal and the other signal and/or a difference of the signal and the other signal.

According to other examples, an apparatus to personalize a laser treatment procedure on a patient is provided. The apparatus may include a detection device and a processor coupled to the detection device. The detection device may be configured to detect an effect of a first laser pulse in response to a treatment site of the patient as part of the laser treatment procedure, and generate a signal based on the detected effect. The processor may be configured to receive the signal from the detection device, determine a classification for the patient based on the received signal, determine an adjustment for a remainder of the laser treatment procedure based on the classification of the patient, and provide the adjustment for the remainder of the laser treatment procedure to a laser treatment system and/or a healthcare personnel.

According to some embodiments, the processor may be configured to determine the classification for the patient based on a determination of a category for the patient based on the effect of the first laser pulse among multiple categories, where the multiple categories are based on collected data from multiple patients. The processor may be configured to determine the adjustment for the remainder of the laser treatment procedure through adjustment of a number of remaining laser pulses, a strength of the remaining laser pulses and/or a timing of the remaining laser pulses. The processor may be configured to determine the adjustment for the timing of the remaining laser pulses through an adjustment of a frequency and/or a duration of the remaining laser pulses.

According to other embodiments, the detection device may be configured to detect the effect of the first laser pulse through acoustic and/or optical detection. The processor may be further configured to compute a similarity metric between the signal and a reference signal and determine the classification for the patient based on the similarity metric. The processor may be configured to compute the similarity metric through a dynamic time warping technique and/or a wavelet technique. The treatment site may be an eye and the processor may be configured to determine the classification for the patient based on the effect of the first laser pulse and a characteristic of the eye. The characteristic may include a size of the eye, an elasticity of the eye, a pressure of the eye, and/or a fluid content of the eye.

According to further embodiments, the processor may be further configured to compute a confidence metric for the classification of the patient based on the signal. The processor may be further configured to receive an other signal from the detection device based on detection of an effect of a second laser pulse in response to application of the second laser pulse to the treatment site of the patient as part of the laser treatment procedure, and determine the classification for the patient based on the signal and the other signal. The processor may be configured to determine the classification for the patient based on an average of the signal and the other signal and/or a difference of the signal and the other signal.

According to further examples, a system is provided to personalize a laser treatment procedure on a patient. The system may include a laser device, a detection device, and a processor coupled to the laser device and the detection device. The laser device may be configured to provide multiple laser pulses to a treatment site of the patient as part of the laser treatment procedure. The detection device may be configured to detect an effect of a first laser pulse at the treatment site in response to application of the first laser pulse by the laser device, and generate a signal based on the detected effect. The processor may be configured to receive the signal from the detection device, determine a classification for the patient based on the received signal, determine an adjustment for a remainder of the laser treatment procedure based on the classification of the patient, and provide the adjustment for the remainder of the laser treatment procedure to the laser device.

According to some embodiments, the processor may be configured to determine the classification for the patient based on a determination of a category for the patient based on the effect of the first laser pulse among multiple categories, where the multiple categories are based on collected data from multiple patients. The processor may be configured to determine the adjustment for the remainder of the laser treatment procedure through adjustment of a number of remaining laser pulses, a strength of the remaining laser pulses, and/or a timing of the remaining laser pulses. The processor may be configured to determine the adjustment for the timing of the remaining laser pulses through an adjustment of a frequency and/or a duration of the remaining laser pulses.

According to other embodiments, the detection device may be configured to detect the effect of the first laser pulse through acoustic and/or optical detection. The processor may be further configured to compute a similarity metric between the signal and a reference signal and determine the classification for the patient based on the similarity metric. The processor may be configured to compute the similarity metric through a dynamic time warping technique and/or a wavelet technique. The treatment site may be an eye and the processor may be configured to determine the classification for the patient based on the effect of the first laser pulse at the treatment site and a characteristic of the eye. The characteristic may include a size of the eye, an elasticity of the eye, a pressure of the eye, and/or a fluid content of the eye.

According to further embodiments, the processor may be further configured to compute a confidence metric for the classification of the patient based on the signal. The processor may be further configured to receive an other signal from the detection device based on detection of an effect of a second laser pulse at the treatment site in response to application of the second laser pulse to the treatment site of the patient by the laser device, and determine the classification for the patient based on the signal and the other signal. The processor may be configured to determine the classification for the patient based on an average of the signal and the other signal and/or a difference of the signal and the other signal.

There are various vehicles by which processes and/or systems and/or other technologies described herein may be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for mainly hardware and/or firmware vehicle; if flexibility is paramount, the implementer may opt for mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, may be equivalently implemented in integrated circuits, as one or more computer programs executing on one or more computers (e.g., as one or more programs executing on one or more computer systems), as one or more programs executing on one or more processors (e.g., as one or more programs executing on one or more microprocessors), as firmware, or as virtually any combination thereof, and that designing the circuitry and/or writing the code for the software and/or firmware are possible in light of this disclosure.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing particular embodiments only, and is not intended to be limiting.

In addition, the mechanisms of the subject matter described herein are capable of being distributed as a program product in a variety of forms, and that an illustrative embodiment of the subject matter described herein applies regardless of the particular type of signal-bearing medium used to actually carry out the distribution. Examples of a signal-bearing medium include, but are not limited to, the following: a recordable type medium such as a floppy disk, a hard disk drive (HDD), a compact disc (CD), a digital versatile disk (DVD), a digital tape, a computer memory, a solid state drive (SSD), etc.; and a transmission type medium such as a digital and/or an analog communication medium (e.g., a fiber optic cable, a waveguide, a wired communication link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. A data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity of gantry systems; control motors to move and/or adjust components and/or quantities).

A data processing system may be implemented utilizing any suitable commercially available components, such as those found in data computing/communication and/or network computing/communication systems. The herein described subject matter sometimes illustrates different components contained within, or connected with, different other components. Such depicted architectures are merely exemplary, and in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

For any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. All language such as “up to,” “at least,” “greater than,” “less than,” and the like include the number recited and refer to ranges which can be subsequently broken down into subranges as discussed above. Finally, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

1. A method to personalize a laser treatment procedure on a patient, the method comprising:
applying a first laser pulse to a treatment site of the patient as part of the laser treatment procedure;
in response to the application of the first laser pulse, receiving a signal based on an observation of an effect of the first laser pulse;
determining, based on the received signal, a classification for the patient;
adjusting, based on the classification of the patient, a remainder of the laser treatment procedure; and
continuing the adjusted remainder of the laser treatment procedure.

2. The method of claim 1, wherein determining the classification for the patient comprises:
determining a category for the patient based on the effect of the first laser pulse among a plurality of categories, wherein the plurality of categories is based on collected data from a plurality of patients.

3. The method of claim 1, wherein adjusting the remainder of the laser treatment procedure comprises:
adjusting one or more of a number, a strength, a timing, a shape, or an energy of remaining laser pulses and/or one or more of a beam profile, a radiance, an intensity, or an energy of an applied laser beam for a completion of the laser treatment procedure.

4. (canceled)

5. (canceled)

6. The method of claim 1, wherein receiving the signal based on the observation of the effect of the first laser pulse comprises:
receiving the signal based on one or more of an acoustic detection, an optical detection, an electromagnetic detection, or an interferometric detection of the effect of the first laser pulse.

7. (canceled)

8. The method of claim 1, wherein determining the classification for the patient comprises:
computing a similarity metric between the signal and a reference signal; and
determining the classification for the patient based on the similarity metric.

9. The method of claim 8, wherein computing the similarity metric comprises:
computing the similarity metric through a dynamic time warping technique or a wavelet technique.

10. (canceled)

11. The method of claim 1, wherein the treatment site is a portion of an eye and determining the classification for the patient further comprises:
determining the category for the patient based on the effect of the first laser pulse and a characteristic of the eye, the characteristic including one or more of a size of the eye, an elasticity of the eye, a pressure of the eye, a fluid content of the eye, a location of a photoceptor cell at the treatment site, a position of the photoceptor cell at the treatment site, a type of the photoceptor cells at the treatment site, an amount of melanin at the treatment site, or a content of stem cells near the treatment site.

12. (canceled)

13. (canceled)

14. The method of claim 1, further comprising:
in response to application of a second laser pulse to the treatment site of the patient as part of the laser treatment procedure, receiving another signal based on the observation of an effect of the second laser pulse; and
determining the classification for the patient based on one or more of an average or a difference of the signal and the other signal.

15. (canceled)

16. (canceled)

17. An apparatus to personalize a laser treatment procedure on a patient, the apparatus comprising:
a detection device configured to:
detect an effect of a first laser pulse in response to application of the first laser pulse to a treatment site of the patient as part of the laser treatment procedure; and
generate a signal based on the detected effect; and
a processor coupled to the detection device, the processor configured to:
receive the signal from the detection device;
determine, based on the received signal, a classification for the patient based on a determination of a category for the patient among a plurality of categories, wherein the plurality of categories is based on collected data from a plurality of patients;
determine an adjustment, based on the classification of the patient, for a remainder of the laser treatment procedure; and
provide the adjustment for the remainder of the laser treatment procedure to one or more of a laser treatment system and a healthcare personnel.

18. (canceled)

19. The apparatus of claim 17, wherein the processor is configured to determine the adjustment for the remainder of the laser treatment procedure through adjustment of one or more of:
a number of remaining laser pulses, a strength of the remaining laser pulses, a shape of the remaining laser pulses, an energy of the remaining laser pulses, a frequency of the remaining laser pulses, a duration of the remaining laser pulses, or a timing of the remaining laser pulses, or
a beam profile, a radiance, an intensity, or an energy of an applied laser beam for a remainder of the laser treatment procedure.

20. (canceled)

21. The apparatus of claim 17, wherein the detection device is configured to detect the effect of the first laser pulse through one or more of acoustic detection, electromagnetic detection, optical detection, or interferometric detection.

22. (canceled)

23. The apparatus of claim 17, wherein the processor is further configured to:
compute a similarity metric between the signal and a reference signal through a dynamic time warping technique or a wavelet technique; and
determine the classification for the patient based on the similarity metric.

24. (canceled)

25. (canceled)

26. The apparatus of claim 17, wherein the treatment site is a portion of an eye and the processor is configured to determine the classification for the patient based on the effect of the first laser pulse and a characteristic of the eye, the characteristic including one or more of a size of the eye, an elasticity of the eye, a pressure of the eye, a fluid content of the eye, a location of a photoceptor cell at the treatment site, a position of the photoceptor cell at the treatment site, a type of the photoceptor cells at the treatment site, an amount of melanin at the treatment site, or a content of stem cells near the treatment site.

27. (canceled)

28. The apparatus of claim 17, wherein the processor is further configured to:
compute a confidence metric for the classification of the patient based on the signal.

29.-31. (canceled)

32. A system to personalize a laser treatment procedure on a patient, the system comprising:
a laser device configured to:
provide a plurality of laser pulses to a treatment site of the patient as part of the laser treatment procedure;
a detection device configured to:
detect an effect of a first laser pulse at the treatment site in response to application of the first laser pulse by the laser device; and
generate a signal based on the detected effect; and
a processor coupled to the laser device and the detection device, the processor configured to:
receive the signal from the detection device;
determine, based on the received signal, a classification for the patient based on a determination of a category for the patient among a plurality of categories, wherein the plurality of categories is based on collected data from a plurality of patients;
determine an adjustment, based on the classification of the patient, for a remainder of the laser treatment procedure; and
provide the adjustment for the remainder of the laser treatment procedure to the laser device.

33. (canceled)

34. The system of claim 32, wherein the processor is configured to determine the adjustment for the remainder of the laser treatment procedure through adjustment of one or more of:
a number of remaining laser pulses, a strength of the remaining laser pulses, a shape of the remaining laser pulses, an energy of the remaining laser pulses, a frequency of the remaining laser pulses, a duration of the remaining laser pulses, or a timing of the remaining laser pulses, or
a beam profile, a radiance, an intensity, or an energy of an applied laser beam for a remainder of the laser treatment procedure.

35. (canceled)

36. The system of claim 32, wherein the detection device is configured to detect the effect of the first laser pulse through one or more of acoustic detection, electromagnetic detection, optical detection, or interferometric detection.

37. (canceled)

38. The system of claim 32, wherein the processor is further configured to:
compute a similarity metric between the signal and a reference signal through a dynamic time warping technique or a wavelet technique; and
determine the classification for the patient based on the similarity metric.

39. (canceled)

40. (canceled)

41. The system of claim 32, wherein the treatment site is a portion of an eye and the processor is configured to determine the classification for the patient based on the effect of the first laser pulse at the treatment site and a characteristic of the eye, the characteristic including one or more of a size of the eye, an elasticity of the eye, a pressure of the eye, a fluid content of the eye, a location of a photoceptor cell at the treatment site, a position of the photoceptor cell at the treatment site, a type of the photoceptor cells at the treatment site, an amount of melanin at the treatment site, or a content of stem cells near the treatment site.

42. (canceled)

43. (canceled)

44. The system of claim 32, wherein the processor is further configured to:
in response to application of a second laser pulse to the treatment site of the patient by the laser device, receive another signal from the detection device based on detection of an effect of the second laser pulse at the treatment site; and
determine the classification for the patient based on an average or a difference of the signal and the other signal.

45. (canceled)

46. (canceled)
